(19) United States
(12) Patent Application Publication
(10) Pub. No.: US 2009/0047369 A1
(43) Pub. Date: Feb. 19, 2009

(54) Composition Comprising 4-Carbon Dicarboxlic Acids for Improvement of Memory Function

(75) Inventor: Jin Kyu Park, Daejeon (KR)

Correspondence Address: STAAS & HALSEY LLP, SUITE 700, 1201 NEW YORK AVENUE, N.W., WASHINGTON, DC, 20005, US

(73) Assignee: Eubioslab Co., Ltd, Daejee (KR)

(21) Appl. No.: 12/223,976

(22) PCT Filed: Feb. 13, 2007
(86) PCT No.: PCT/KR2007/000760

§ 371 Date: Aug. 14, 2008

(30) Foreign Application Priority Data
Feb. 18, 2006 (KR) 10-2006-0015921
Feb. 12, 2007 (KR) 10-2007-0014175

(51) Int. Cl. A61K 31/194; A61K 36/736

(52) U.S. Cl. 424/735; 514/574

(57) ABSTRACT

Disclosed herein is a composition for the improvement of memory and learning functions, which contains 4-carbon dicarboxylic acid or a salt thereof and an emulsifying agent. The composition shows a more remarkable effect when it additionally contains a concentrate or alcohol extract of maesil. Because the composition improves learning and memory functions, it is effective for the prevention and treatment of amnesia or dementia and can also be applied as a health supplement food or functional food for this prevention and treatment.

### TECHNICAL FIELD

The present invention relates to a composition for the improvement of learning and memory functions, which contains 4-carbon dicarboxylic acid as an active ingredient.

### BACKGROUND ART

Learning capabilities, including memory, are central processes having decisive effects on the daily life and activity of modern persons, and include a series of mental processes, for example, attention, short term memory, long term memory, reasoning, co-ordination of movement, planning of tasks and so on. Also, learning capabilities usually influence directly the mental activity of modern persons, and ultimately, have an effect on the quality of life. It is known that continuous mental stress or depression is a major cause of reducing the attention and memory of normal persons, and in severe cases, impairs the function of brain hippocampus and causes defects in neuropsychic functions.

There is now a consensus that the human hippocampus is involved in episodic memory (memory of events or episodes that one experienced personally at a particular time and place). Likewise, it is out of the question that the hippocampus is associated with spatial or positional memory in animals other than human beings(Neuron, 2002; 35(4), 625-641). A change in the hippocampus has a close connection with learning, memory, emotional control or the like. The hippocampus shows synaptic plasticity in response to various paradigms including Long Term Potentiation(LTP)(Nature, 1993; 361, 31-39) of which spatial training in the Morris water maze has been amongst the most intensively investigated(New York: Oxford University Press. 1978, Nature, 1982; 297, 681-683).

CREB(Cyclic AMP response element binding protein), P-CREB(phosphorylated CREB) that are induced in hippocampus signal transduction plays an important role in the induction of anti-depressant effects. When CREB is induced in the hippocampus by treatment with anti-depressant drugs, it can contribute to therapeutic effects, whereas drug addiction mediated by drug abuse or stress, mental depression, anxiety and the like activate CREB in the nucleus accumbens and other sites of the brain. In particular, the CREB in hippocampus influences memory and learning. It was reported that CREB is one of transcription factors and performs an important role in the formation of long term memory by influencing memory and learning in various animal models(Cell, 1994; 79, 59-68). Moreover, among various kinases involved in the phosphorylation of CREB, ERK(extracellular-signal regulated kinase) is a regulator of CREB phosphorylation, which performs an important role in memory and synaptic plasticity.

The activation of ERK in the brain hippocampus is associated with synaptic plasticity and is required for the formation and enhancement of memory through various types of learning in mammals, and CREB is well known as a transcription factor performing an important role in the formation of long-term memory. If an antisense CREB oligonucleotide is injected into the brain hippocampus, followed by performing a Morris water-maze test, the test animal will show a deficiency in long-term memory(Proc. Natl. Acad. Sci. USA 1997; 94, 2693-2698), CREB phosphorylation will be increased in the CA1 region of hippocampus when LTP is induced(Neuron 1996; 16, 89-101, Behavioral Brain Research, 2002; 133, 135-141). BDNF mRNA in the hippocampus will be increased during the induction of LTP or after a radial maze test (Neuron 1992; 9, 1081-1088). BDNF is a member of the neurotrophin family, which has an intimate effect on synaptic plasticity, and when the expression of BDNF is inhibited by an antisense BNDF oligonucleotide, an impediment in the acquisition and maintenance of learning will occur(J. Neurosci. 2000; 20, 7116-7121). Moreover, TrkB is a BNDF receptor, which is activated by BDNF(Life Sciences, 2002; 70, 735˜744), is a factor important for the maintenance of memory together with NMDAR(NMDA receptor, NR1, NR2A, NR2B), and is necessary for the synaptic plasticity of the hippocampus CAI area(Nature Neuroscience, 2002; 5, 27-33).

Succinic acid(SA) and fumaric acid(FA) are intermediate metabolites of tricarboxylate cycle(TCA cycle) and are typical compounds playing a very important role in the energy metabolism of brain mitochondria. In the case of fumaric acid, when it was administered after hypoxia, it showed nerve protective effects. This protective effect was considered as a result of the action of fumaric acid away from the SDH step of the TCA cycle when the activity of SDH(Succinate dehydrogenase) in ischemia-induced brain was significantly inhibited(Ann. Thorac. Surg. 1994; 57, 1636-1641). However, there is still no study on the effect of fumaric acid on the improvement of memory and learning capabilities in normal conditions.

Saarma J. et al. (Int. Pharmaco psychiatry, 1975; 10(3), 149-156) examined higher nerve activity after administering each of SSA(succinic semialdehyde) and SS(sodium succinate) to normal persons. As a result, they suggested that SSA enhances the excitability of the cerebral cortex to increase verbal system activity, whereas SS has the possibility of psychoenergizer that stabilizes the excitability of the cerebral cortex.

Meanwhile, in the case of a thiamine-deficient model, memory deficit and neurological disease were induced not only in human beings, but also animals. This is similar to the initial symptom of Alzheimer's disease, in that it shows a nerve injury, such as amyloid precursor protein-like immune activity, and an initial cholinergic deficit. Bubber et al. (Neurochem. Int. 2004; 45(7), 1021-1028) examined variations in TCA cycle enzymes in the brain of mice supplied with thiamine-deficient diet. The study results showed that the activities of SDH and STH (succinic acid thiokinase), which are not thiamine-dependent enzymes, were significantly decreased compared to a control group. This phenomenon was suggested to be important in understanding a change in brain function, which occurs in neurodegenerative disease. Such results suggest that the administration of each of fumaric acid, succinic acid and the like can be involved in the improvement of neurodegenerative disease associated with impairment in memory and learning.

The dicarboxylic acids of the TCA cycle are known to be very limited with respect to passage through the blood-brain barrier (BBB). Thus, the way by which dicarboxylic acids influence the brain can also be limited. However, stress conditions influence the state of the brain hippocampus according to the regulation of CRF (corticotrophin releasing factor) by HPA-axis rather than BBB, anti-depressant drugs can inhibit steroid transporters in BBB and neuron in depression patients and animal and cell models, and the inhibitory mechanism of the steroid transporters is associated with negative feed back of corticosterone via HPA-axis, which is increased due to endogenous glucocorticoids.

Accordingly, it is expected that, when anti-depressant drugs are administered in combination with dicarboxylic acids in a normal condition for a given period of time, they can have a positive effect on the plasticity-memory ability of the brain hippocampus.

Maesil(Japanese Apricot, mume, plum) is known as one of health supplement foods in Korea and Japan and receives attention as a food material for the prevention of cardiovascular diseases, such as thrombosis, a reduction in neutral lipid, and blood pressure lowering associated with the inhibition of angiotensin converting enzyme(ACE). However, there is still no example where Maesil extracts, having effects on the blood flow enhancement, the promotion of absorption in the stomach or intestines, the regulation of blood pressure, the mitigation of climacteric stress, etc., were effectively used in the improvement of memory and learning functions.

Drugs developed for the improvement of memory and learning functions to date include γ-aminobutyric acid derivatives, acetylcholine precursors, receptor agonists and nicotine, which act to increase the metabolism of the central nervous system, and also Tacrine and Aricept (donepezil), which act to improve recognition function by preventing the decomposition of neurotransmitters. However, these drugs are known to have temporary effects and to cause serious side effects. In addition, emulsifying surfactants such as tween80 or Span80 effective in the brain have problems associated with passage through the blood-brain barrier, and thus the verification of effects of dicarboxylic acids with the nonionic detergents through in vivo experiments is required in order to solve the problems. However, active studies on this verification are not yet conducted.

### DISCLOSURE

### Technical Problem

The present invention has been made in order to solve the above-described problems and it is an object of the present invention to provide a composition for the improvement of memory and leaning functions, which has no side effects and is safe.

Another object of the present invention is to provide a composition for the improvement of memory and learning functions, which can be actually used as an agent for the improvement of memory and learning functions by solving a problem in that the availability of 4-carbon dicarboxylic acid in the brain is reduced due to low blood-brain barrier permeability.

Still another object of the present invention is to provide a composition for the improvement of learning and memory functions, which can be used as a pharmaceutical composition for the improvement of brain function, such as the treatment of amnesia, and also health supplement food or functional food.

### Technical Solution

To achieve the above objects, the present invention provides a composition for the improvement of memory and learning functions, which contains 4-carbon dicarboxylic acid or a salt thereof and an emulsifying agent.

The 4-carbon dicarboxylic acid is one or a mixture of two or more selected from the group consisting of fumaric acid, succinic acid and salts thereof. The salts of 4-carbon dicarboxylic acid include all acceptable salts, but preferably include alkali metals Li, Na and K. Also, the salts of 4-dicarboxylic acid include, in addition to metal salts, complexes with CoA-S (coenzyme A synthetase).

The dosage of 4-carbon dicarboxylic acid (or salt thereof) as an agent for the improvement of learning and memory functions varies depending on the age, bodyweight and disease severity of a patient, but it is preferably administered at a dosage of 300-900 mg/day three times for adults weighing 60 kg. It may be administered in an amount of 100-500 mg per each administration.

4-carbon dicarboxylic acids such as fumaric acid and succinic acid are well known with respect to the toxicity and safety thereof and have been approved as food additives. Thus, the 4-carbon dicarboxylic acids can be safely used without causing any side effects.

As the emulsifying agent, Tween 80 (polyoxyethylene sorbitan monooleate) and Span 80 (sorbitan monooleate) were used in Examples of the present invention, but the scope of the present invention is not limited thereto. In addition to Tween 80 and Span 80, the emulsifying agent may be any one or a mixture of two or more selected from the group consisting of bile acid, glycerin, glycerin fatty acid ester, propylene glycol, sucrose, sorbitol, sodium cholate, sorbitan monolaurate, sorbitan monopalmitate, sorbitan tristearate, sorbitan monostearate, polyoxyethylene sorbitan monolaurate, polyoxyethylene sorbitan monopalmitate, polyoxyethylene sorbitan monostearate, deoxycholate, sodium taurocholate, lecithins and lysolecithins.

The emulsifying agent is preferably administered at an average dosage of 75-1800 mg/day in an amount of 25-600 mg per each administration for adults. In the composition according to the present invention, the mixing ratio of the 4-carbon dicarboxylic acid: the emulsifying agent may be within the range of 1:0.05˜5.

The composition, comprising the 4-dicarboxylic acid and the emulsifying agent, may further comprise a concentrate or alcohol extract of plum (maesil in Korean language).

The maesil concentrate is a concentrate of maesil juice, which is prepared by heating maesil juice at 40˜50° C. to remove water. In the present invention, a concentrate obtained by concentrating maesil juice to a final water content of 14±3% was used, but the scope of the present invention is not limited thereto, and the water content of the maesil concentrate can be changed, if necessary. The maesil concentrate can be prepared and used as the need arises, but when it is stored for a long period of time before use, fungi or the like can occur during the storage thereof. For this reason, the maesil concentrate is placed into a bottle and subjected to flash pasteurization at 110-120° C. before storage.

The alcohol extract of maesil is prepared by adding alcohol and water to maesil, followed by extraction. As alcohol, it is preferable to use lower alcohol such as methanol, ethanol, propanol or butanol, and it is more preferable to use ethanol in consideration of the toxicity of the remaining solvent. In the extraction solvent, the volume ratio of alcohol to water is preferably 50:50 to 100:0. More preferably, the extraction solvent contains 60-90 vol % of alcohol.

The concentrate or alcohol extract of maesil preferably contains malic acid and fumaric acid at concentrations of 3-15% and 0.3-10%, respectively, but the scope of the present invention is not limited thereto, because the concentrations of the acids can be changed depending on conditions and concentration extent in the concentration or extraction process. When the concentrate or extract of maesil is used in the composition together with the 4-carbon dicarboxylic acid and the emulsifying agent, the weight ratio of the concentrate or alcohol extract of maesil: the 4-carbon dicarboxylic acid: the emulsifying agent is preferably 1-3: 1:0.05-5, but it will be obvious to one skilled in that art that the mixing ratio and dosage of the components can be determined in consideration of the content of each of organic acids and water in the concentrate or alcohol extract of maesil.

Hereinafter, the present invention will be described in detail.

In the present invention, the effect of formulated drugs on the improvement of learning and memory of a normal test animal group (Sprague Dawley rats) was tested by administering the drugs to the test animals via an oral or drinking water route for 2, 3 or 3 weeks and performing a Morris water maze test at 2, 3 or 3 weeks of the drug administration.

In the Morris water maze test, each of the test animals was given four swimming trials per session per day for 4-5 days. When the escape latency of the test animal, that is, the time for the test animal to reach a hidden platform 2 cm below the surface of water, was shortened to about 10±2 seconds during the repeat of the learning sessions, a probe test was performed after 24 hours one time for 60 seconds without the platform.

Meanwhile, when the significance between the control group and the test group was not shown during the learning sessions, memory for new learning was tested by performing a short-term working memory test instead of the probe test. In this case, the difference in memory index between the control group and the drug-administered group was not significant, average escape latency shown as a result of a short-term memory test was defined as “adaptability to new relearning” for comparison between the control group and the drug-administered group.

Rats in the growth stage were administered with a composition, comprising 4-carbon dicarboxylic acid or its salt and an emulsifying agent, and the learning and working memory abilities of the rats was measured with a Morris water-maze test and a probe test or short-term working memory test. As a result, the group administered with the composition showed a significant improvement compared to the control group (see FIGS. 1 to 4). Thus, it can be seen that the composition, comprising 4-carbon dicarboxylic acid or its salt and an emulsifying agent, is effective as a composition for the improvement of learning and memory functions. However, when 4-carbon dicarboxylic acid was administered alone without an emulsifying agent, it had no significant effect on the improvement of learning and memory functions (data not shown).

Depression is generally characterized in that memory and attention are markedly reduced, and a maesil(plum) extract has an anti-depressant effect. In consideration of these facts, a test was performed to examine whether a combination of a concentrate or alcohol extract of maesil with the dicarboxylic acid of the TCA cycle can enhance attention and improve blood flow so as to show a synergy effect on the improvement of memory and learning functions.

An anti-depressant effect was tested using a tail suspension test (TST), which is most generally used for the screening of anti-depressant drugs(Steru L, Chermat R, Thierry B, Simon P., Psycopharmacology 1985, 85(3), 367-370). In TST, an antidepressant effect as a decrease in immobility time caused by antidepressant drugs was tested by suspending the tail of test animals and measuring each of the agitation time and immobility time of the animals during the test period. From the TST results, it was confirmed that an alcohol extract of maesil had an anti-depressant effect, but when it was used alone, it had no effect on the improvement of learning and memory functions (see FIGS. 5 and 6).

Compositions comprising a mixture of an alcohol extract of maesil with succinic acid and/or an emulsifying agent were tested with respect to the improvement of learning and memory functions. As a result, the administration of compositions comprising a mixture of an alcohol extract of maesil with succinic acid and/or an emulsifying agent showed a more significant and effective anti-depressant effect compared with the case where the alcohol extract of maesil was administered alone (see FIGS. 7 and 8). In the case where a maesil concentrate was used instead of the alcohol extract of maesil, a significant antidepressant effect compared to the control group was observed (see FIG. 9).

A composition comprising a mixture of an alcohol extract of maesil with succinic acid did not show significant results in a working memory ability test, but showed a significant improvement in “adaptability to new relearning” in a short-term memory ability test (see FIGS. 10 and 11). This suggests that 4-carbon dicarboxylic acid and an alcohol extract of maesil showed a synergy effect compared to results (data not shown) showing that when 4-carbon dicarboxylic acid was administered alone without an emulsifying agent to normal test animals, it did not show a significant improvement in memory and learning functions despite long-term administration (4 weeks).

A composition comprising a mixture of an alcohol extract of maesil with succinic acid and an emulsifying agent showed a significant difference from the control group in both a Morris water-maze test and a probe test (see FIGS. 12 and 13). In Examples of the present invention, only the results of use of Span80 as an emulsifying agent were described, but the same results could also be obtained when Tween80 was used instead of Span80 (data not shown).

A composition comprising fumaric acid instead of succinic acid as 4-carbon dicarboxylic acid could show the same effect as that of a composition comprising succinic acid (see FIG. 14).

A composition, comprising a maesil concentrate, succinic acid and an emulsifying agent, showed a significant difference from the control group in both Morris water-maze test and probe test in the same manner as the composition comprising the alcohol extract of maesil. Particularly, the use of sodium succinate instead of succinic acid showed a significant effect on the improvement of memory and learning functions even when the dosage of an emulsifying agent was reduced (see FIGS. 15 to 17). Although test results were not shown, a mixture of a maesil concentrate with succinic acid, to which an emulsifying agent was not added, did not show a significant effect on the improvement of memory and learning in the same manner as the alcohol extract of maesil, suggesting that the composition comprising the emulsifying agent had a synergy effect.

The contents of glucose, GOT, GPT and cholesterol in serum were analyzed after administration of a maesil concentrate, succinic acid or a salt thereof and Span80. As a result, a great change in each of indexes was not observed (FIG. 18) and there was no significant change in bodyweight and diet between test groups.

To confirm the safety of the composition according to the present invention, the composition was administered orally one time, followed by observation for 14 days. As a result, the composition showed no lethality even at dosages of 2 g/kg and 4 g/kg (based on the content of sodium succinate in the composition). Also, neither significant change in body weight nor a specific necropsy finding compared to a normal control group was observed (data not shown).

Moreover, in the case of test animals, which showed a significant difference in a probe test, the brain hippocampus was isolated and the mRNA expression levels of memory and learning-related signaling substances (e.g., CREB, NMDAR (NR1, NR2A, NR2B), ERK1/2 and trKB) in the isolated hippocampus were examined comparatively with the control group in order to analyze the improvement of learning and memory function of the composition-administered group in a point of view of molecular signaling mechanisms. As a result, the composition-administered group showed a significant increase in the mRNA expression levels of TrkB and NR2B compared to the control group, but there was no difference in other mRNA expression levels between the test group and the control group. Also, the phosphorylation of ERK1/2 and CREB, which are important in memory-related signal transduction, was analyzed. As a result, the amounts of the ERK1/2 and CREB proteins were not changed, whereas the phosphorylation of these proteins was significantly increased compared to the control group (see FIG. 20). This suggests that a memory-related signaling process can be significantly activated to enhance synaptic plasticity.

As described above, it could be the composition according to the present invention showed a more excellent synergy effect on the improvement of learning and memory functions when the components thereof are administered together compared to when each of the components thereof are independently administered. Thus, the inventive composition comprising the above-described components can be used for commercial purposes as an effective material for the improvement of memory and learning.

In order for the inventive composition for the improvement of memory and learning to be used as a therapeutic or preventive drug or food, it can be formulated according to any method known in the pharmaceutical or food industries. It can be prepared per se or in the form of formulations, such as powders, granules, tablets, capsules or injection solutions, by mixing it with, for example, a pharmaceutically or food-acceptable carrier, forming agent or diluent. Also, it can be formulated into unit dosage formulations or multiple dosage formulations, such as formulations for oral or parenteral administration, which can be used as therapeutic and preventive drugs having an effect on the improvement of memory and learning.

The pharmaceutical composition according to the present invention is formulated into formulations for oral administration, for example, troches, lozenges, aqueous or oily suspensions, dispersible powders or granules, emulsions, hard or soft capsules, syrups or elixirs. To make a formulation of tablets or capsules, a binding agent such as lactose, saccharose, sorbitol, mannitol or cellulose, an excipient such as dicalcium phosphate, glycerin, fatty acid ester, propylene glycol, purified processed oils, soybean lecithin, palm oil or beewax, a disintegrating agent such as starch or gelatin, a lubricant such as stearate or polyethyleneglycol wax, a flavoring agent, a sunscreen, and a coloring agent, are used. The capsule formulation contains, in addition to the above-mentioned substances, a liquid carrier such as fatty oil.

Furthermore, the pharmaceutical composition of the present invention can be administered orally or parenterally. The parenteral administration of the composition is performed by subcutaneous injection, intravenous injection, intramuscular injection or intrathoracic injection. To make formulations for parenteral administration, the composition for the improvement of memory or learning is mixed with a stabilizer or a buffer in water to prepare a solution or suspension, which is then manufactured into a unit dosage form of ampoules or vials.

### ADVANTAGEOUS EFFECTS

When the inventive composition, comprising 4-carbon dicarboxylic acid and an emulsifying agent (and an alcohol extract of maesil(MS) or a concentrate of maesil(MSM)), is continuously administered to test animals for a given period of time, it will provide an anti-depressant effect for the test animals and also effectively improve the spatial recognition learning and working memory of the test animals.

### DESCRIPTION OF DRAWINGS

FIG. 1 is a graphic diagram showing the test results of spatial learning and working memory, obtained by administering a composition of fumaric acid-Tween80 to normal test animals for 4 weeks and performing a Morris water-maze test and a probe test.

FIG. 2 is a graphic diagram showing the test results of spatial learning and memory for new learning, obtained by administering a composition of fumaric acid-Tween80 to normal test animals for 2 weeks and performing a Morris water-maze test and a probe test.

FIG. 3 is a graphic diagram showing the test results of spatial learning and memory for new learning, obtained by administering a composition of succinic acid-Tween80 to normal test animals for 4 weeks and performing a Morris water-maze test and a short-term working memory test.

FIG. 4 is a graphic diagram showing the test results of spatial learning and memory for new learning, obtained by administering a composition of succinic acid-Span80 to normal test animals for 3 weeks and performing a Morris water-maze test and a probe test.

FIG. 5 is a graphic diagram showing the results of analysis of an antidepressant effect, obtained by administering an alcohol extract of maesil to normal test animals for 3 weeks and performing TST.

FIG. 6 is a graphic diagram showing the test results of spatial learning and memory for new learning, obtained by administering a composition of an alcohol extract of maesil to normal test animals for 4 weeks and performing a Morris water-maze test and a short-term working memory test.

FIG. 7 is a graphic diagram showing the results of analysis of an antidepressant effect, obtained by administering a composition, comprising an alcohol extract of maesil and succinic acid and/or Span80, to normal test animals for 1 week and performing TST.

FIG. 8 is a graphic diagram showing the results of analysis of an antidepressant effect, obtained by administering a composition, comprising an alcohol extract of maesil and succinic acid, to normal test animals for 3 weeks and performing TST.

FIG. 9 is a graphic diagram showing the results of analysis of an antidepressant effect, obtained by administering a composition, comprising a maesil concentrate and succinic acid disodium salt, to normal test animals for 3 weeks and performing TST.

FIG. 10 is a graphic diagram showing the test results of spatial learning and memory for new learning, obtained by administering a composition, comprising an alcohol extract of maesil and succinic acid, to normal test animals for 3 weeks and performing a Morris water-maze test, a probe teat and a short-term working memory test.

FIG. 11 is a graphic diagram showing the test results of spatial learning and memory for new learning, obtained by administering a composition, comprising an alcohol extract of maesil and succinic acid, to normal test animals for 4 weeks and performing a Morris water-maze test, a probe teat and a short-term working memory test.

FIG. 12 is a graphic diagram showing the test results of spatial learning and memory for new learning, obtained by administering a composition, comprising an alcohol extract of maesil, succinic acid and Span80, to normal test animals for 3 weeks and performing a Morris water-maze test and a short-term working memory test.

FIG. 13 is a graphic diagram showing the test results of spatial learning and memory for new learning, obtained by administering a composition, comprising an alcohol extract of maesil, succinic acid and Span80, to normal test animals for 2 weeks and performing a Morris water-maze test and a short-term working memory test.

FIG. 14 is a graphic diagram showing the test results of spatial learning and memory for new learning, obtained by administering a composition, comprising an alcohol extract of maesil, fumaric acid and Span80, to normal test animals for 3 weeks and performing a Morris water-maze test and a short-term working memory test.

FIG. 15 is a graphic diagram showing the analysis results of spatial learning and memory for new learning, obtained by administering a composition, comprising a maesil concentrate, succinic acid or a salt thereof and Span80, to normal test animals through drinking water for 3 weeks and performing a Morris water-maze test and a short-term working memory test.

FIG. 16 is a graphic diagram showing the test results of spatial learning and memory for new learning, obtained by orally administering a composition, comprising a maesil concentrate, succinic acid or a salt thereof and Span80, to normal test animals for 3 weeks and performing a Morris water-maze test and a short-term working memory test.

FIG. 17 is a graphic diagram showing the test results of spatial learning and memory for new relearning, obtained by performing a short-term working memory test following the tests of FIG. 16.

FIG. 18 is a graphic diagram showing test results obtained by orally administering a composition, comprising a maesil concentrate, succinic acid or a salt thereof and Span80, to normal test animals for 3 weeks and analyzing the contents of glucose, GOT, GPT and cholesterol in serum.

FIG. 19 is a graphic diagram showing test results obtained by administering a composition, comprising a maesil concentrate, succinic acid disodium salt and Span80, to normal test animals for 3 weeks, performing a Morris water-maze test and a probe test, and quantifying the mRNA of ERK1, ERK2, CREB, TrKB, NR1, NR2A and NR2B in the brain hippocampus of the test animals.

FIG. 20 is a graphic diagram showing test results obtained by administering a composition, comprising a maesil concentrate, succinic acid disodium salt and Span80, to normal test animals for 3 weeks, performing a Morris water-maze test and a probe test, and quantifying changes in ERK, pERK, CREB and pCREB proteins in the brain hippocampus of the test animals.

### BEST MODE

Hereinafter, the present invention will be described in further detail with reference to examples. It is to be understood, however, that these examples are illustrative only and the scope of the present invention is not limited thereto.

In the present invention, the effect of formulated drugs on the improvement of learning and memory for spatial recognition of a normal test animal group (Sprague Dawley rats) was tested by administering the drugs to the test animals orally or through drinking water for 2, 3 or 4 weeks and performing a Morris water maze test at 2, 3 or 4 weeks after the drug administration.

In the Morris water maze test, each of the test animals was given four swimming trials per session per day for 4-5 days. When the escape latency of the test animal, that is, the time for the test animal to reach a hidden platform 2 cm below the surface of water, became shorter than 10±2 seconds during the repeat of the learning sessions, a probe test was performed after 24 hours one time for 60 seconds without the platform. The probe test was performed by removing the platform from the swimming pool and observing the swimming behavior of the test animals. In the probe test, as measures of spatial working memory, the retention time and movement distance of the test animals in the same area of a quadrant region including the position in which the platform was present during the first learning period were analyzed and compared with those of the control group.

Meanwhile, when the significance between the test group and the control group was not shown during the Morris mater maze test, a short-term working memory test was performed instead of the probe test to analyze memory for new learning. In the short-term working memory test, the ability of the test animals to find a newly learned platform was examined by transferring the platform from the position in the first learning period to the opposite quadrant and performing 3 or 4 swimming trails. In this case, the difference between first latency time and second latency time measured after moving the platform to the second position was expressed as memory index, and when the difference in memory index between the control group and the drug-administered group was not shown to be significant, average latency time shown as a result of a short-term working memory test for new relearning was defined as “adaptability to relearning”.

In each of animal tests, the name of each group will be defined using the English abbreviations of administered components. English abbreviations used to indicate the components are as follows: FA: fumaric acid; SA: succinic acid; SS: sodium succinate; MS: an alcohol extract of maesil (plum); MSM: a concentrate of maesil(plum); Tw: Tween80; Sp: Span80; and DW: distilled water. When various components are administered at the same time, the compositions will be listed using the abbreviations thereof. For example, when fumaric acid and Tween80 are administered at the same time, they will be described as FATw. The dosage of each of components was based on the bodyweight of each of test animals, and each of components was dissolved in purified water, and then administered orally or through drinking water ad libitum in an amount per kg of bodyweight. The amount (mg) of each component administered per kg of bodyweight of each test animal was described as a subscript on the name of each composition. More specifically, a test animal group administered with 100 mg of fumaric acid per kg of bodyweight was described as FA₁₀₀, and a test animal group administered with 100 mg of fumaric acid together with 200 mg of Span80 was described as FA₁₀₀Sp₂₀₀. Unless otherwise mentioned in the following examples, the name of each group will indicate the components and dosage of the composition administered to the group.

### EXAMPLE 1

### Effect of Administration of Composition, Comprising Fumaric Acid and Tween80, on Learning and Memory Functions

1) Administration for 4 Weeks

Twenty-one 6-week-old rats, each weighing 180-200 g, were divided into the following three groups, each consisting of 7 animals: a Tw₅₀ group (control group); a FA₅₀Tw₅₀ group; and a FA₁₀₀Tw₁₀₀ group. Each of the compositions was administered to the rats through drinking water for 4 weeks.

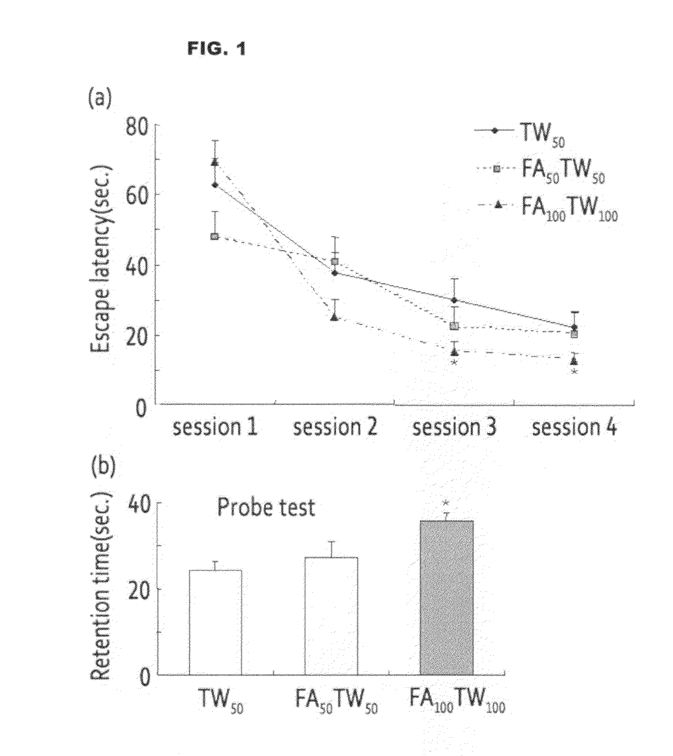

Starting from 4 weeks of the administration, a Morris water maze test was performed at a given time point daily four times per day for 4 days to analyze the effect of each composition on the improvement of learning and memory for spatial recognition function of the test animals. As the test results are shown in FIG. 1(a) showing the escape time of each group.

At 5 days, that is, after 24 hours when the escape time for the learned test animals to reach the platform became an average of 10±2 seconds, the platform was removed and a probe test was performed one time. The test results are shown in FIG. 1(b) to analyze an improvement in learning and memory for spatial recognition functions.

The administration of fumaric acid to the normal rats showed a dose-dependent effect on the improvement of learning and memory. In particular, when it was administered at a dose of 100 mg/kg, it showed a significant (p<0.05) effect on the improvement of both learning and working memory compared to the control group.

2) Administration for 2 Weeks

Twenty-one 6-week-old rats, each weighing 180-200 g, were divided into the following three groups, each consisting of 7 animals: a Tw₁₀₀ group (control group); a FA₁₀₀Tw₁₀₀ group; and a FA₂₀₀Tw₂₀₀ group. Each of the compositions was administered to the rats through drinking water for 2 weeks.

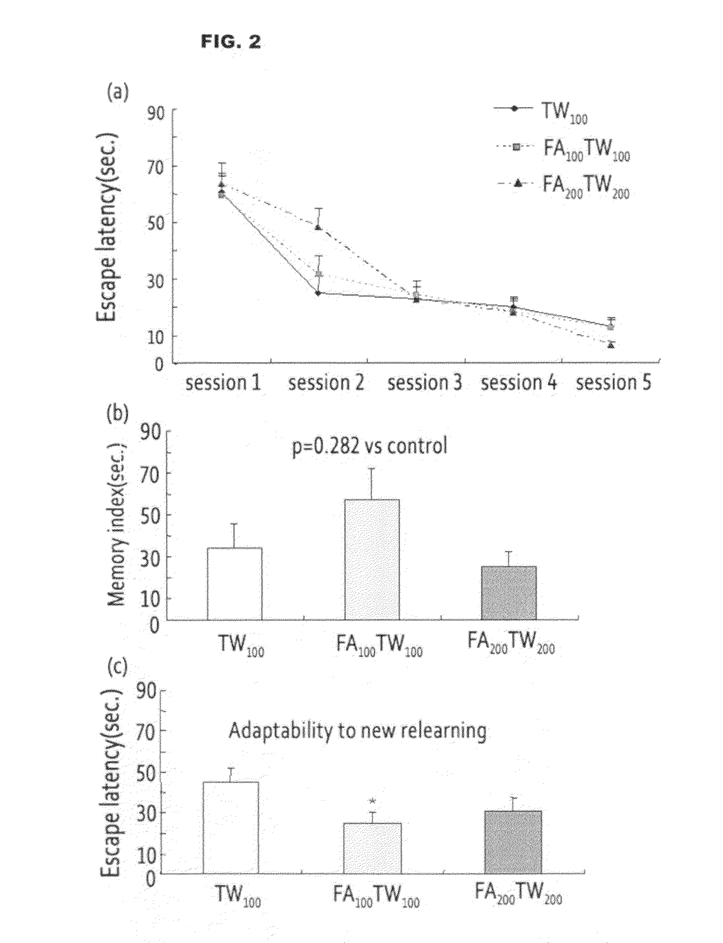

Starting from 2 weeks of the administration, a Morris water maze test was performed at a given time point daily four times per day and as a result, the escape time of each group is shown in FIG. 2(a). As can be seen in FIG. 2(a), at 5 days (session 5) of the test, the escape time of the FA₁₀₀Tw₁₀₀ group was significantly decreased.

At 6 days of the test, the platform was moved to the opposite quadrant, the test animals were allowed to start from the same position, and the time for the test animals to find the moved platform. The test results are graphically shown in FIG. 2(c). Herein, the difference between the time taken to reach the first platform and the time taken to reach the second platform was defined as short-term working memory, and the rate of increase relative to the control group was expressed as memory index. The test results of memory index are shown in FIG. 2(b). The FA₁₀₀Tw₁₀₀ group showed a significant improvement in both the memory index and the escape time in the second learning, and the FA₂₀₀TW₂₀₀ group showed a higher memory index for short-term working.

### EXAMPLE 2

### Effect of 4-Week Administration of Composition, Comprising Succinic Acid and Tween80, on Learning and Memory Functions

Twenty-one 6-week-old rats, each weighing 180-200 g, were divided into the following three groups, each consisting of 7 animals: a Tw₂₀₀ group; a SA₁₀₀Tw₁₀₀ group; and a SA₂₀₀Tw₂₀₀ group. Each of the compositions was administered to the rats through drinking water for 4 weeks.

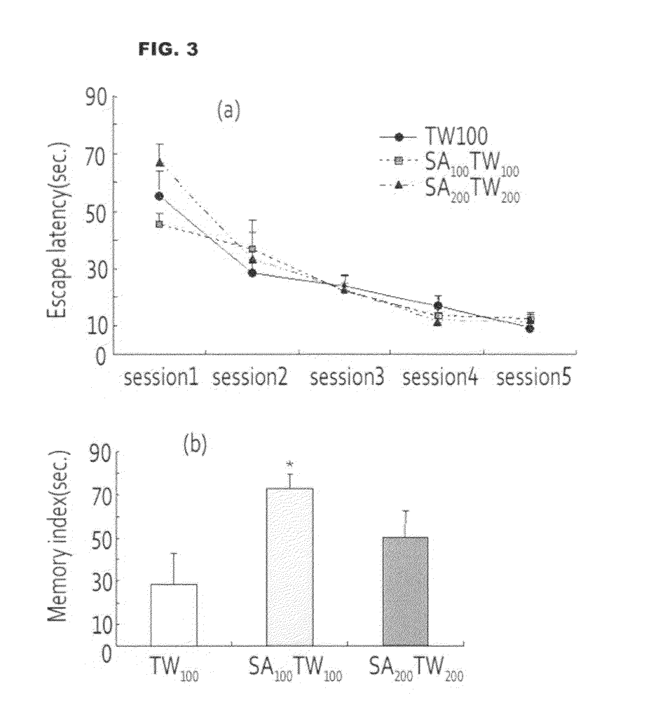

Starting from 4 weeks of the administration, a learning and working memory test, and then short-term working memory test were performed in the same manner as in Example 2 in the Morris water maze. The test results are shown in FIGS. 3(a) and 3(b), respectively. The groups administered with succinic acid did not show a difference in learning ability in the Morris water maze test, but the memory index for short-term working memory was significantly improved in the case of the SA₁₀₀Tw₁₀₀ group.

### EXAMPLE 3

### Effect of 3-Week Administration of Composition, Comprising Succinic Acid and Span80, on Learning and Memory Functions

Span80 was used instead of Tween80 to test the effect thereof on the improvement on learning and memory. Specifically, fourteen 6-week-old rats, each weighing 180-200 g, were divided into the following two groups, each consisting of 7 animals: a DW group; and a SA₁₀₀Sp₂₀₀ group. Each of the compositions was administered to the rats through drinking water for 3 weeks.

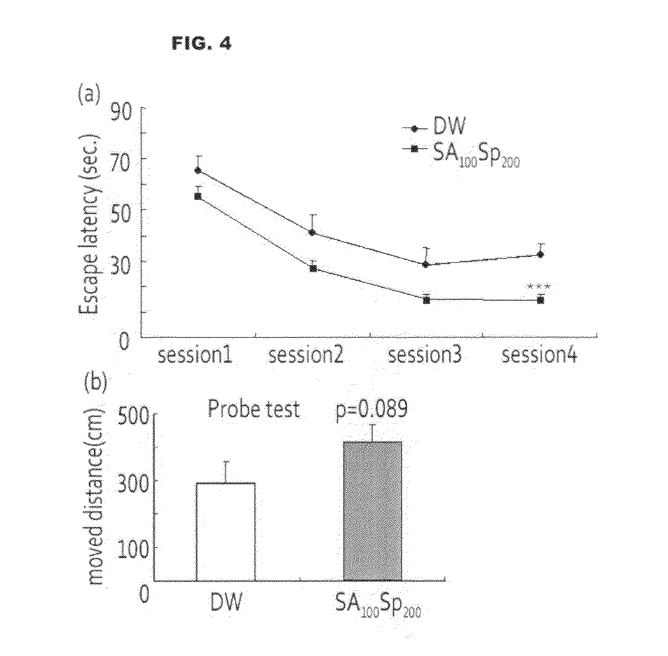

Starting from 3 weeks of the administration, a Morris water maze learning and probe trial were performed in the same manner as in Example 1. The test results are shown in FIGS. 4(a) and 4(b), respectively. The group administered with succinic acid showed a significant increase in learning ability in the Morris water maze test (session 4; *: P<0.001 vs control in Mann Whitney U test). However, in the probe test results, the group administered with succinic acid showed an insignificant (P=0.089) improvement in working memory shown as a result of learning.

### EXAMPLE 4

### Preparation of Alcohol Extract of Maesil (MS)

In the middle of May 2005, 100 kg of maesil was purchased, washed clean with tap water, dried for 2 hours and placed in a pot. Then, an aqueous alcohol solution was prepared by mixing 22.5 liters of water with 90 liters of 95% alcohol, and placed in the pot such that the plum(maesil) was sufficiently immersed. The pot was made airtight and left to stand at room temperature for 3 months. After 3 months, the maesil was separated out, a portion (about 45 liters) of the remaining alcohol liquor was concentrated in a vacuum at 55° C. to obtain about 625 g of a final concentrate. The concentrate was used in subsequent animal tests.

For the standardization of an alcohol extract of maesil, each of the contents of succinic acid, fumaric acid and malic acid in the extract was quantified with a gas chromatograph using standards.

For quantification, each of 1.2 mg, 1.4 mg of fumaric acid and 1.2 mg of malic acid was collected from the sample, and 100 μl of N,O-bis(trimethylsilyl)acetamide (Aldrich Chemical Company) was added thereto. Each of the mixtures was allowed to react in an oven at 70° C. for 60 minutes. 900 μl of CHCl₃ (HPLC grade) was added to each of the reaction materials, and the resulting mixtures were used as analytical samples. 3 μl of each of the trimethylsilylated sample solutions was collected and analyzed with GC (GC-14B, Shimadzu, Japan). As a detector, FID (flame ionization detector) was used, and as a column, DB-5HP (30 m×0.32 mm ID×0.1 μm film thickness; J&W, Folsom, Calif., USA) was used. The oven temperature was elevated from 100° C. at a rate of 15° C./min and maintained at a final temperature of 320° C. for 5 minutes. The injector port and the detector port were all maintained at a temperature of 300° C. Each of the tests was repeated three times and the test results were analyzed. As a result, the regression equation of fumaric acid was y=420921x−5921.2 (r=0.9995), and the regression equation of succinic acid was y=265256x+22047 (r=0.9981). As a result of substitution into each of the regression equations, the maesil extract had a malic acid content of 13.4% and a fumaric acid content of 8% (2.55 mg/31.6 mg), and no succinic acid was detected in the maesil extract.

### EXAMPLE 5

### Effect of Alcohol Extract of Maesil on Improvement of Depression and Learning Ability

To analyze the antidepressant effect according to dose of an alcohol extract of maesil MS), ICR mice (male, 20±5 g) were divided into five groups, each consisting of 20 animals. The alcohol extract of maesil was administered to the animal groups at varying doses for 3 weeks (experiment repeated two times), followed by TST.

Specifically, the test groups were administered orally with 100, 200, 400 and 800 mg/kg of the alcohol extract of maesil(MS) and distilled water at the same time point daily one time per day for 3 weeks. At 1 hour after administration at day 21, the last day of 3 weeks, 1-2 cm of the tail end of each mouse was suspended to the corner of a 80-cm-height horizontal plane using a strongly adhesive tape, and then recorded on a video recorder for 5 minutes. Based on a time of 4 minutes other than the first 1 minute, the immobility time of the test animals was measured using the recorded video tape.

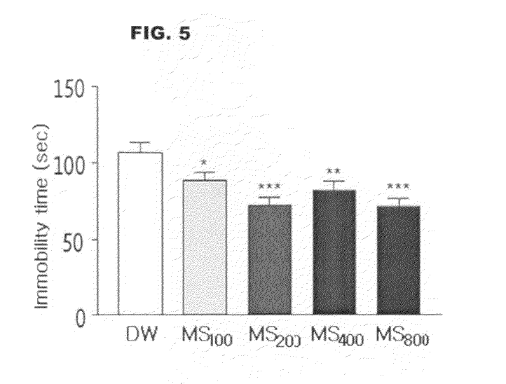

The results of TST at varying MS doses are shown in FIG. 5. As can be seen in FIG. 5, the alcohol extract of maesil (MS) showed a significant difference from the control group in the dose range of 100-800 mg/kg b.w., although the difference was not dose-dependent. Particularly, the group administered with 200 mg of the alcohol extract of maesil showed a distinct significance compared to the control group (control vs mean difference 34.94, q 5.896, P<0.001).

In order to confirm the effect of administration of MS on the improvement of memory and learning functions, twenty rats, each weighing about 200 g, were divided into a DW group (control group) and a MS₂₀₀ group, each group consisting of 10 animals. The control group was administered only with distilled water, and in the case of the MS₂₀₀ group, MS dissolved in distilled water was administered to the rats ad libitum through drinking water for 4 weeks.

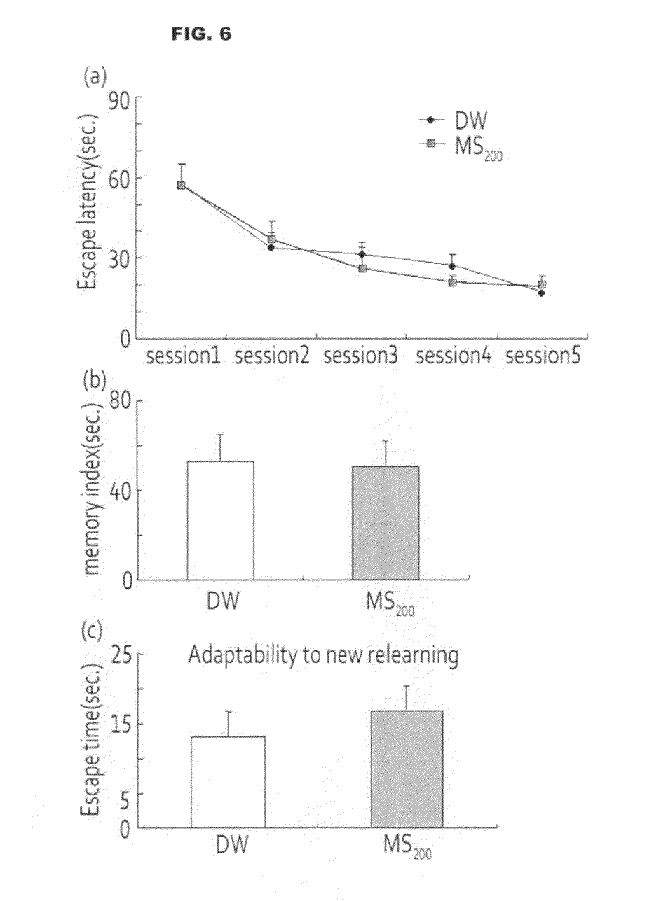

After 4 weeks, escape time in a Morris water maze test, memory index in a short-term working memory test, and adaptability to secondary learning, were measured in the same manner as in Example 2, and the measurement results are shown in FIGS. 6(a), 6(b) and 6(c), respectively. As can be seen in FIG. 6, MS₂₀₀ did not show a significant effect on the learning ability and working memory of the test animals during the learning period even when it was administered for 4 weeks.

### EXAMPLE 6

### Effect of Administration of Composition, Comprising Alcohol Extract of Maesil or Maesil Extract and Succinic Acid and/or Span80, on Improvement of Depression

A test was performed to examine whether the combined administration of MS₂₀₀ that showed an antidepressant effect and succinic acid that had the effect of enhancing learning ability but showed an insignificant effect on the enhancement of the significance of working memory, and Span80, showed an effect on the improvement of memory and learning functions.

To examine the antidepressant effect caused by the combined administration of MS, succinic acid and Span80, a tail suspension test (TST) was performed on ICR mice (male).

Hundred ICR mice, each weighing 20-25 g, were divided into a control group, an MS₂₀₀ group, an MS₂₀₀SA₁₀₀ group, an MS₂₀₀SA₂₀₀ group and an MS₂₀₀SA₁₀₀SP₂₀₀ group, each consisting of 20 animals. Each of the composition was administered orally to the mice one time per day for 1 week. The control group was administered with purified water. Separately, twenty ICR mice were divided into a control group and an imipramine (Im) group, each consisting of 10 animals, and were administered intraperitoneally with distilled water and 10 mg/kg of imipramine (Im), respectively, one time per day for 1 week.

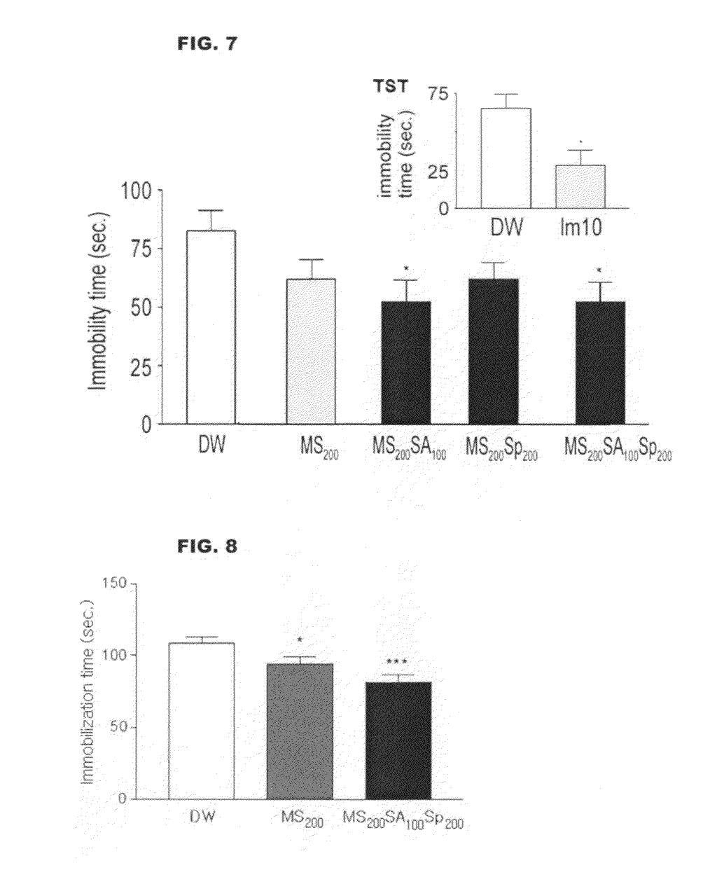

Imipramine is one of tricyclic antidepressants and a typical drug of the family that inhibits serotonine uptake, and it shows a significant antidepressant effect when it is administered for 1 week(Jpn J Psychiatry Neurol 1991; 45, 113-114). After 1 week, a TST test was performed two times in the same manner as in Example 4, and the test results are shown in FIG. 7. As can be seen in FIG. 7, the antidepressant effect of MS was more remarkable when it was administered together with succinic acid and/or Span80. MS is a functional food, which showed an effect lower than that of single drug imipramine. However, it showed a remarkable antidepressant effect when it was administered for 3 weeks or longer. Also, the administration of MS in combination with succinic acid (p<0.05, q 2.896) or in combination with succinic acid and Span80 showed a more significant antidepressant effect (p<0.001, q 5.285) vs control in Newman-Keuls multiple comparison test) compared to when it was administered alone.

In order to examine whether the composition shows the same effect even when it is administered for a long period of time, sixty ICR mice were divided into DW group (control group), MS₂₀₀ group and MS₂₀₀SA₁₀₀SP₂₀₀ group, each consisting of 20 animals, and allowed for free access to drinking water containing each of the compositions for 3 weeks. Then, a TST test was repeated two times, and the test results are shown in FIG. 8. The antidepressant effects of the MS₂₀₀ group and the MS₂₀₀SA₁₀₀Sp₂₀₀ group were shown to be significant even when they were administered orally every day for 3 weeks, and the administration of MS in combination of succinic acid and Span80 showed a more significant (P<0.05) antidepressant effect compared to when it was administered alone.

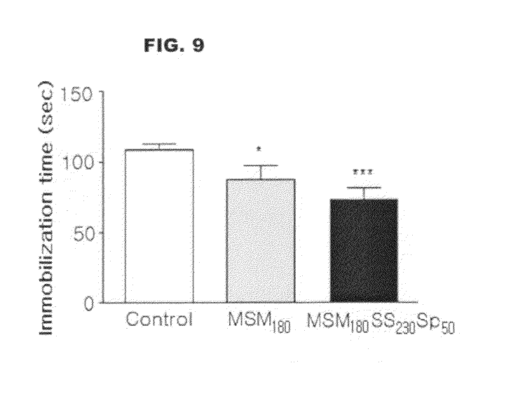

In order to examine whether a maesil concentrate also has an antidepressant effect similar to that of the alcohol extract of maesil, sixty ICR mice, each weighing 20-25 g, were divided into a control group (DW group), an MSM₁₈₀ group and an MSM₁₈₀SS₂₃₀Sp₅₀ group, each consisting of 20 animals, and were administered orally with each of the compositions daily one time for 3 weeks. Then, a TST test was repeated two times, and the test results are shown in FIG. 9. As can be seen in FIG. 9, like the alcohol extract of maesil, the MSM₁₈₀SS₂₃₀Sp₅₀ group administered with the maesil concentrate in combination with succinic acid and Span80 showed a more significant (P<0.05) antidepressant effect compared to the MSM₁₈₀ group administered with the maesil concentrate alone.

### EXAMPLE 7

### Effect of Administration of Composition, Comprising Alcohol Extract of Maesil and Succinic Acid, on Improvement of Memory and Learning Functions

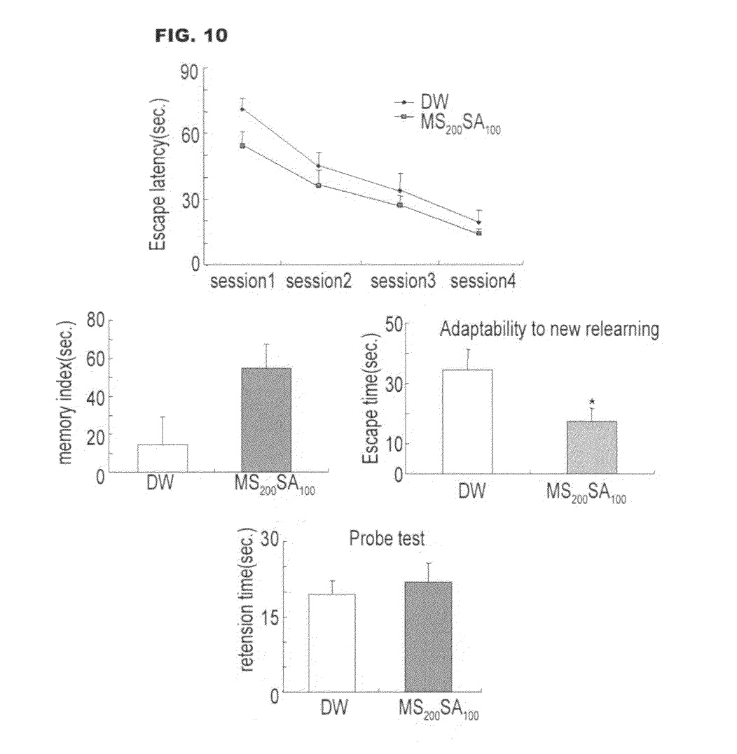

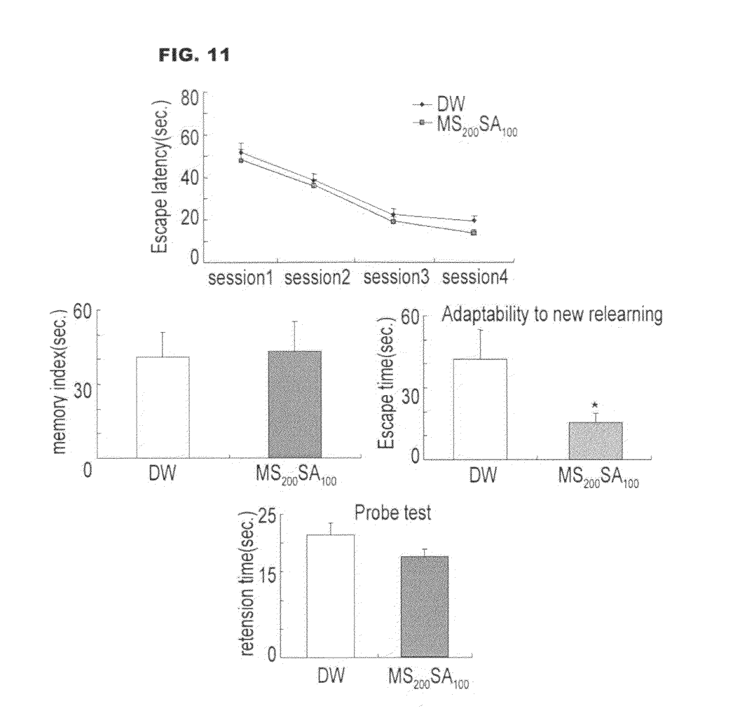

Forty rats, each weighing 200 g, were divided into a 3-week-administration group and a 4-week-administration group, each consisting of 20 animals. Each of the group was subdivided into a DW group and an MS₂₀₀SA₁₀₀ group. The 3-week-administration group was administered with each of the compositions through drinking water for 3 weeks, and the four-week-administration group was administered with each of the compositions through drinking water for 4 weeks. Then, the test animals were subjected to a Morris water maze test, a probe test and a short-term working memory test. The test results for the 3-week-administration group are shown in FIG. 10, and the test results for the 4-week-administration group are shown in FIG. 11. In FIGS. 10 and 11, (a) shows the results of the Morris water maze test, (b) shows the results of the probe test, (c) shows memory index caused by the short-term working memory test, and (d) shows adaptability to secondary learning. In the case of 3-week-administration, significant results were not obtained in the working memory test, but adaptability to new relearning was significantly enhanced in the short-term working memory caused by the movement of the platform. In the case of 4-week administration, the working memory in the probe test did not a significant difference, but adaptability to new relearning for the moved platform was effectively improved. This suggests that the administration of MS₂₀₀SA₁₀₀ enhances adaptability to new relearning by enhancing “attention to new learning” and resistance to stress compared to when SA was administered alone.

### EXAMPLE 8

### Effect of Administration of Composition, Comprising alcohol extract of masil, organic acid and Span80, on Improvement of Memory and Learning Functions

1) 3-Week Administration of Alcohol Extract of Maesil, Succinic acid and Span80

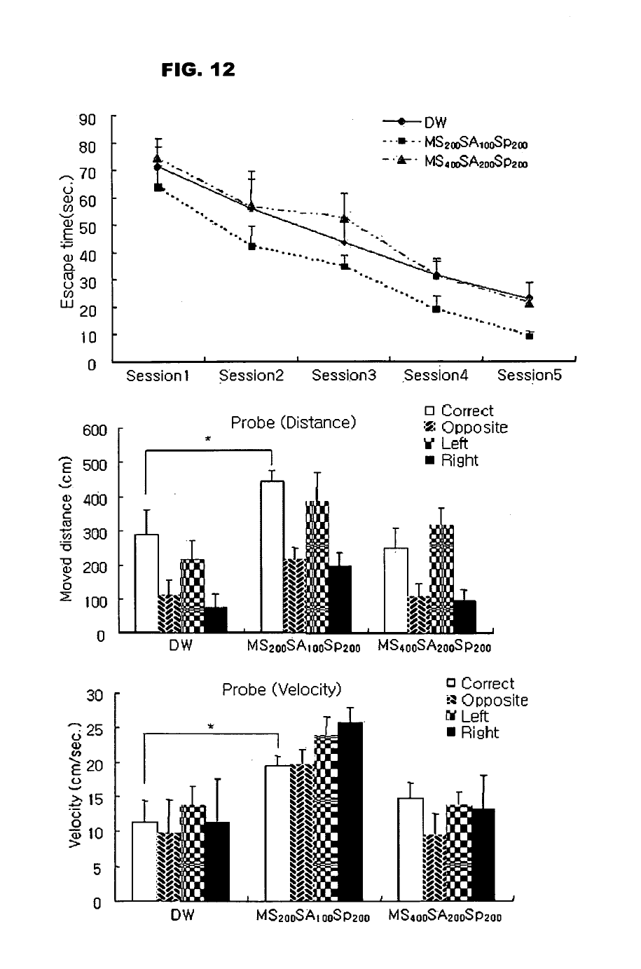

In Order to Examine the Effect of the Combined administration of MS, succinic acid and Span80 on the improvement of memory and learning functions, twenty-one 6-week-old rats, each weighing 180-200 g, were divided into a DW group, an MS₂₀₀SA₁₀₀Sp₂₀₀ group and an MS₄₀₀SA₂₀₀Sp₂₀₀, each consisting of 7 animals. The rats were administered with each of the compositions through drinking water for 3 weeks, and a Morris water maze test was performed four times per day for 5 days starting from the third week. At 24 hours after the final learning, a probe test was performed to observe an effect on working memory (see FIG. 12).

The MS₂₀₀SA₁₀₀Sp₂₀₀ composition showed significance in the session 5 of the Morris water maze test and also showed a significant improvement in working memory in the probe test. Specifically, in the probe test conducted after learning, the movement distance per unit time of the test animals in a target quadrant in which the platform has been present was increased, suggesting that the spatial recognition memory of the test animals for the position of the platform was improved.

2) 2-Week Administration of Alcohol Extract of Maesil, Succinic Acid and Span80

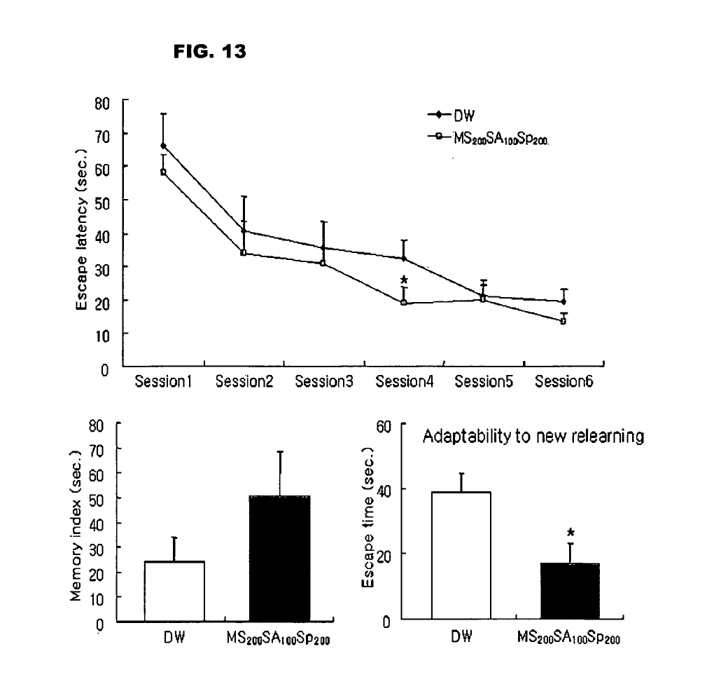

In order to examine the suitable administration period of MS₂₀₀SA₁₀₀Sp₂₀₀, fourteen 6-week-old rats, each weighing 180-200 g, were divided into a DW group and an MS₂₀₀SA₁₀₀Sp₂₀₀ group, each consisting of 7 animals, and were administered with the composition through drinking water for 2 weeks. Starting from the second week, a Morris water maze test was performed for 5 days. At 24 hours after the final water maze test, the platform was moved to the opposite quadrant, and short-term working memory for a new platform was comparatively observed. FIG. 13(a) shows the results of the Morris water maze test, FIG. 13(b) shows memory index resulting from the short-term working memory test, and FIG. 13(c) shows adaptability to secondary learning. In the case of 2-week administration, adaptability to new relearning was significantly improved, but there was no significant increase in memory index that indicates short-term memory. Thus, the effect of 2-week administration on learning and memory functions was lower than that of 3-week or longer administration.

3) 3-Week Administration of Alcohol Extract of Maesil, Fumaric Acid and Span80

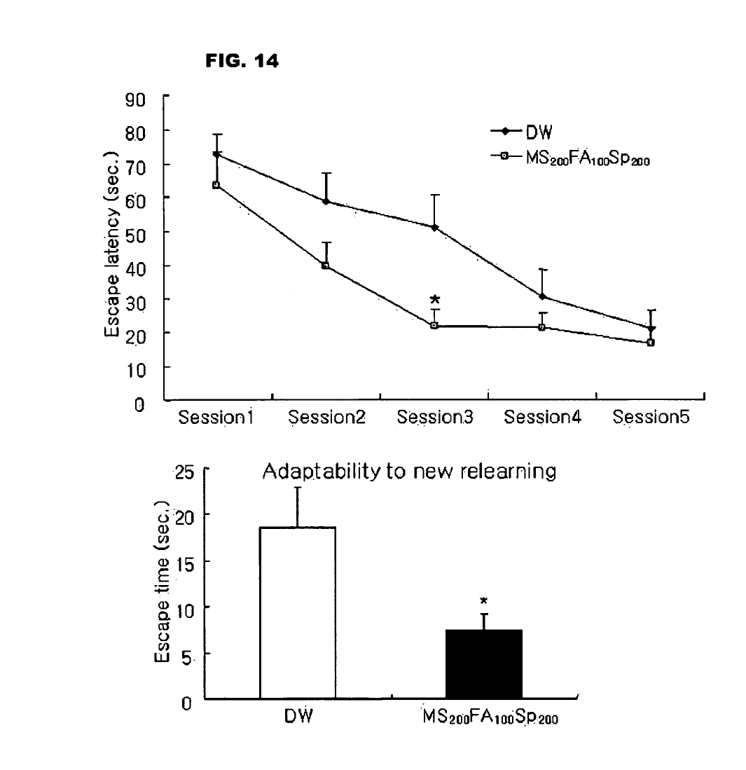

In order to examine the effect of a composition comprising fumaric acid instead of succinic acid, fourteen 6-week-old rats, each weighing 180-200 g, were divided into a DW group and an MS₂₀₀FA₁₀₀Sp80₂₀₀ group, each consisting of 7 animals, and were administered with the composition through drinking water for 3 weeks. Starting from the third week, a Morris water maze test was performed for 5 days. At 24 hours after the final water maze test, the platform was moved to the opposite quadrant, and short-term working memory for a new platform was comparatively observed. FIG. 14(a) shows the results of the Morris water maze test, and FIG. 14(b) shows adaptability to secondary learning.

Like the case of the composition comprising succinic acid in the above section 2), there was no significant increase in memory index that indicates short-term memory, but adaptability to new relearning was significantly improved. Thus, it is expected that the composition, comprising an alcohol extract of maesil, fumaric acid and an emulsifying agent, will also be effective in the improvement of learning and memory functions when the administration period thereof is adjusted.

### EXAMPLE 9

### Effect of Administration of Composition, Comprising Maesil Concentrate, Organic Acid and Span80, on Improvement of Memory and Learning Functions

1) Preparation of Maesil Concentrate

maesil fruits before full maturity were purchased, and among them, 10 kg of maesil having no defect was selected, washed clean with service water, dried, and then ground on a steel sheet or in a mixer, thus making maesil juice. The maesil juice was placed in a stainless container or a ware bowl, in which it was concentrated by heating with slow stirring at 40-50° C. The concentration process was performed until the green juice became a sticky dark brown liquid, thus preparing about 200 g of a maesil concentrate having a water content of 14±3%.

The prepared concentrate was placed in a sterilized bottle in a hot state, cooled, stored airtight, and taken out of the bottle, if necessary.

2) Quantification of Marker Substances of Maesil Concentrate

The contents of fumaric acid and malic acid in the maesil concentrate were quantified according to the quantification method of Example 4. The contents of fumaric acid and malic acid detected in the maesil concentrate were 0.3-10% and 3-15%, respectively, although the contents showed variations depending on the prepared maesil concentrates, because the contents of malic acid and fumaric acid are influenced by the habitat and collection time of prepared maesil concentrates and the atmospheric conditions of harvested maesil. Also, an aqueous solution of a formulation containing the maesil concentrate was heated at 60° C. for 1 hour, insoluble aggregation was observed unlike the alcohol extract of maesil, suggesting that the maesil concentrate and the alcohol extract of maesil were different with respect to the components thereof due to the difference between the preparation methods thereof.

3) Administration of MSM-SA (or SS)-SP Through Drinking Water

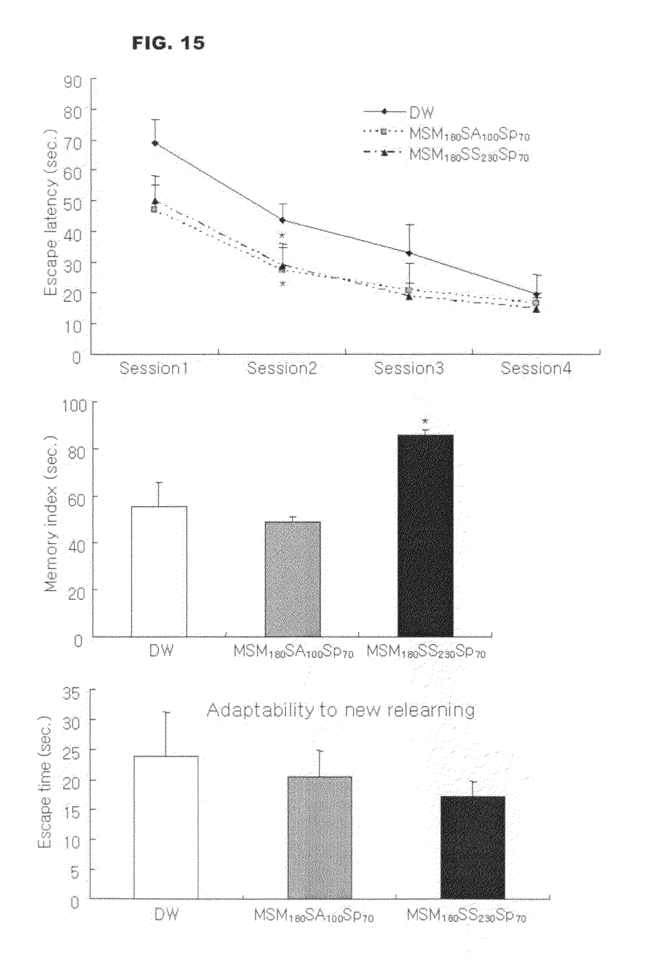

In order to examine the effects of the processing of maesil and the use of a salt of organic acid on the improvement of memory and learning functions, twenty-one 6-week-old rats, each weighing 180-210 g, were divided into a DW group, an MSM₁₈₀SA₁₀₀Sp₇₀ and an MSM₁₈₀SS₂₃₀Sp₇₀ group, each consisting of 7 animals, and were administered with each of the compositions through drinking water for 3 weeks. After 3 weeks, escape time in a Morris water maze test, memory index in a short-term working memory test, and adaptability to secondary learning, were measured in the same manner as in Example 2. The test results are shown in FIGS. 15(a), 15(b) and 15(c), respectively.

As can be seen in FIG. 15, the MSM₁₈₀SA₁₀₀Sp₇₀ group and the MSM₁₈₀SS₂₃₀Sp₇₀ group all showed improved adaptability not only to primary learning, but also to new relearning, although there was no statistical significance in the improvement. Particularly, a composition comprising 230 mg of sodium succinate (SS) having the same number of moles of 100 mg of succinic acid showed substantially the same effect as the administration of a succinic acid-containing composition on adaptability to primary and secondary learning, but the memory index of the group administered with the composition was significantly greatly increased, suggesting that it was more effective than the MSM₁₈₀SA₁₀₀Sp₇₀ composition.

4) Oral Administration of Maesil Concentrate, Succinic Acid (or its Salt) and Span80

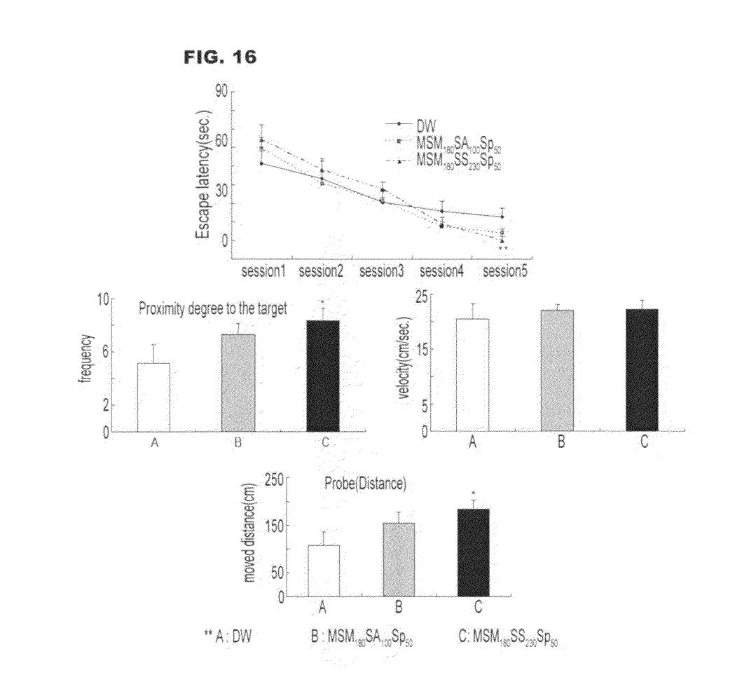

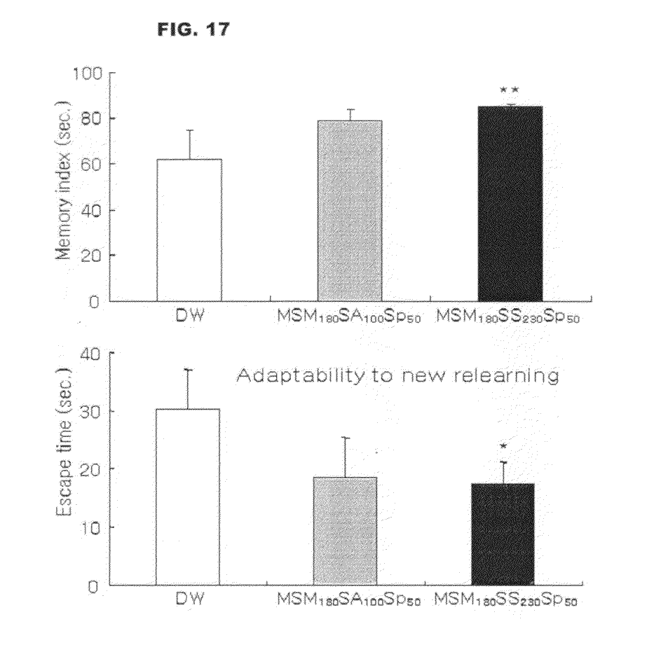

In order to examine the effect of oral administration of a composition on the improvement of memory and learning functions, twenty-one 6-week-old rats, each weighing 180-210 g, were divided into a DW group, an MSM₁₈₀SA₁₀₀SP₅₀ and an MSM₁₈₀SS₂₃₀Sp₅₀ group, each consisting of 7 animals, and were administered with each of the compositions daily one time for 3 weeks. After 3 weeks, a Morris water maze test was performed in the same manner as in Example 2, and the test results are shown in FIG. 16. Also, memory index in a short-term working memory test, and adaptability to secondary learning, were measured and the test results are shown in FIG. 17.

As can be seen in FIGS. 16 and 17, the group administered with MSM₁₈₀SS₂₃₀Sp₅₀ showed a very significant effect on the improvement of learning. Also, in the probe test, the working memory of the group was improved and adaptability to a new learning target was significantly activated. Thus, the group showed an effect on the improvement of short-term working memory corresponding to new learning.

### EXAMPLE 10

### Effect of Administration of Composition, Comprising Maesil Concentrate (or its Salt) and Span80, on Liver Metabolism

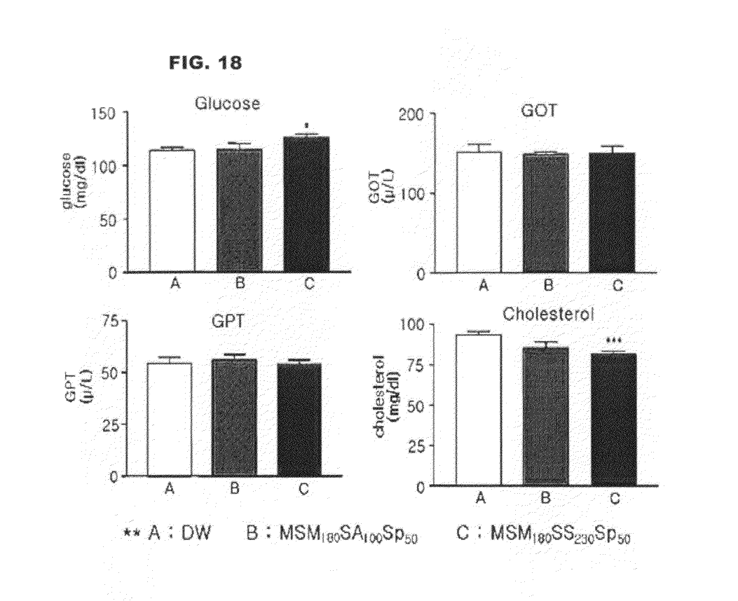

MSM₁₈₀SA₁₀₀Sp₅₀ or MSM₁₈₀SS₂₃₀Sp₅₀ was administered for 3 weeks, and then a Morris water maze test, a probe test and a test for adaptability to new learning were performed. Within 30 minutes after performing the tests, serum was collected, and the contents of glucose, GOT, GPT and cholesterol in the serum were analyzed. The analysis results are shown in FIG. 18. As can be seen in FIG. 18, the content of cholesterol was decreased compared to that of the normal control group, whereas the content of glucose was slightly increased, but was within 15% of the normal range. Also, a significant change in bodyweight and diet quantity was not observed during the administration period.

### EXAMPLE 11

### Analysis of Change in Brain Signal Transduction Substances of Test Animals by 3-Week Administration of composition, comprising maesil concentrate, Succinic Acid (or its Salt) and Span80

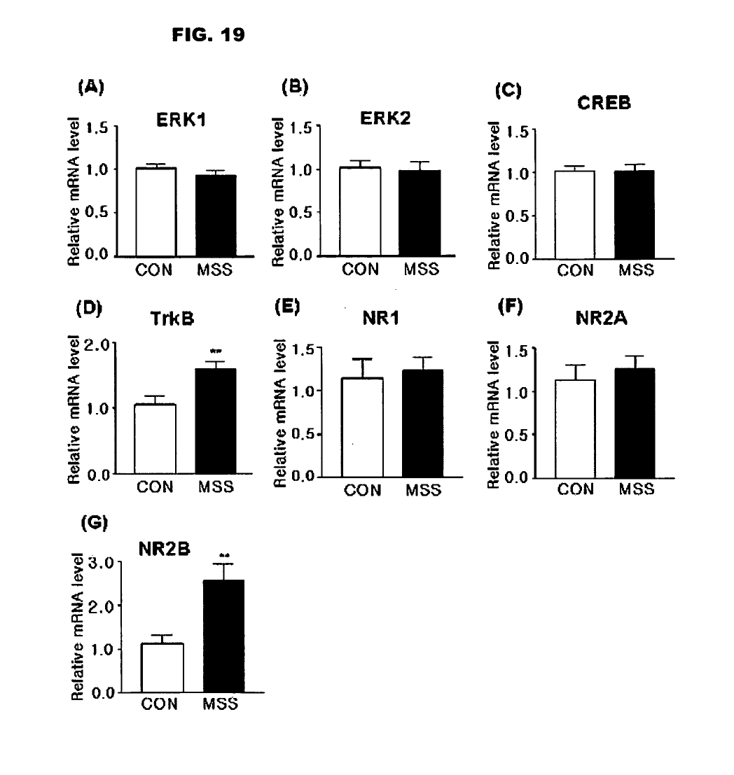

In order to analyze behavior analysis results obtained by the Morris water maze test following the 3-week administration of MSM₁₈₀SS₂₃₀Sp₅₀ in Example 9(4) in a point of view of a molecular signal transduction mechanism by synaptic plasticity, the brain hippocampus of the test animals was isolated after the final probe test, RNA was isolated from the brain hippocampus, and then cDNA was constructed from the isolated RNA. Using the cDNA as a template, real-time PCR was performed using primers of NR1, NR2A, NR2B, ERK1, ERK2, TrkB, CREB and BDNF, and the mRNA copy level relative to GAPDH was calculated. The test results are shown in FIG. 19. In FIG. 19, control represents the control group, and MSS represents the MSM₁₈₀SS₂₃₀Sp₅₀ group.

Specifically, total RNA was isolated from the hippocampus tissue of rats using a Mini RNA Isolation II kit (Zymo Research Co.) and subjected to RT-PCR to synthesize cDNA. Then, the cDNA was subjected to real-time PCR (ABI PRISM 7900 Sequence Detection System) to obtain an amplified PCR product of each of the genes. For the amplification of each of the genes, the Primer3 software was used to design primer pairs shown in Table 1 below. Each of the PCR reaction mixtures consisted of 1×sybr Green PCR Master Mix (Applied Biosystems), 50 nM specific primer sets and 50 ng cDNA, the volume of the resulting reaction was 20 μl, and the PCR reaction was performed in the following conditions: 10 minutes at 95° C., and then 40 cycles, each consisting of 15 sec at 95° C. and 1 min at 60° C. The real-time amplification data were analyzed using the ABI PRISM 7900 SDS Software, thus obtaining threshold cycle (Ct) values. The relative quantity for each of the samples was normalized with GAPDH and substituted into a quantification equation (Methods, 2001; 25(4), 402-408)) to calculate the relative amount for each gene (mRNA).

TABLE 1

| gene | | |
|---|---|---|
| (rat) | Primer sequence (5′-3′) | Seq. No. |
| NR1 for | AGC GGG TAA ACA AGA GGA AGA | Seq. No. 1 |
| NR1 rev | TCC GCT TGG CCA CTG AGT | Seq. No. 2 |
| NR2A for | TGC TTT CCT CGA ACC CTT CA | Seq. No. 3 |
| NR2A rev | CTG AGA CGA TGA GCA GCA TCA | Seq. No. 4 |
| NR2B for | GTG AGA GAT GGA ATT GCC ATC A | Seq. No.5 |
| NR2B rev | GGC TCA GGG ATG AAA CTG TGT T | Seq. No.6 |
| ERK1 for | ACC TGC TGG ACC GGA TGT T | Seq. No. 7 |
| ERK1 rev | GGG TGA GCC AGT GCT TCC T | Seq. No. 8 |
| ERK2 for | TGC TTT CTC TCC CGC ACA A | Seq. No. 9 |
| ERK2 rev | GCT TTG GAG TCA GCG TTT GG | Seq. No. 10 |
| TrkB for | AAT TGT GGA TTC CGG CTT AAA | Seq. No. 11 |
| | GT | |
| TrkB rev | CCG CAG GTT GCC GTT CT | Seq. No. 12 |
| BDNF for | CCA TAA GGA CGC GGA CTT GT | Seq. No. 13 |
| BDNF rev | GAG GCT CCA AAG GCA CTT GA | Seq. No. 14 |
| CREB for | GAA ACA GCT TCT TCT GGC TCA | Seq. No. 15 |
| | TAA A | |
| CREB rev | CAG CAG TAT GAT AGC CAG ACT | Seq. No. 16 |
| | TCT TC | |
| GAPDH for | TGC CAA GTA TGA TGA CAT CAA | Seq. No. 17 |
| | GAA G | |
| GAPDH rev | AGC CCA GGA TGC CCT TTA GT | Seq. No. 18 |

As a result, as can be seen in FIG. 19, the mRNA levels of TrkB and NR2B were significantly increased compared to those of the control group, but there was no difference in the mRNA levels of other genes between the test group and the control group.

Also, the phosphorylation of ERK1/2 and CREB, which are important in memory-related signal transduction, was analyzed. To isolate the proteins from the hippocampus tissue of rats, the hippocampus tissue was added to a lysis buffer, comprising 150 mM sodium chloride (Sigma), 1% Nonidet P-40 (Fluka), 0.5% sodium deoxycholic acid (Sigma), 0.1% sodium dodecyl sulfate (Amersham Biosciences), 50 mM Tris-HCl (pH 7.5, Sigma), 1% protease inhibitor cocktail (Sigma) and 1% phosphatase inhibitor cocktail (Sigma), to make a homogenous solution, which was then centrifuged to collect the supernatant. The protein concentration was quantified using a protein quantification kit (Bio-Rad). The quantified proteins were taken in the same amount, electrophoresed on 10% SDS-PAGE gel and transferred to a PVDF (polyvinylidene difluoride) membrane. The membrane, to which the proteins were transferred, was shaken in a skim milk/TBS blocking buffer for 1 hour to block non-specific antigens. Then, the membrane was allowed to react with primary antibodies, ERK, phosphor-ERK (1:1000, Santa Cruz), CREB and phospho-CREB (1:500, Upstate) at 4° C. overnight. After the membrane was washed three times with TBS, it was allowed to react with secondary antibodies at room temperature for 1 hour, followed by color development with a chemiluminescence kit (Amersham Bioscience). After the film was scanned, the band of each of the proteins was comparatively analyzed using the “Quantity One” software (Bio-Rad). Quantification of each of the samples was performed with reference to the invariant cytoskeletal protein, β-actin, and expressed as a percentage relative to the control group.

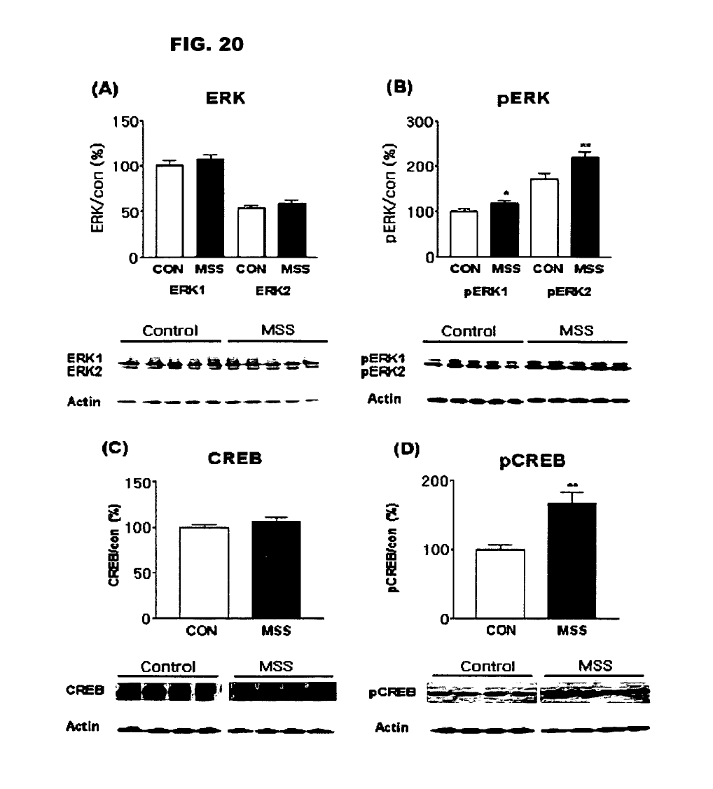

As a result, the amounts of the ERK1/2 and CREB proteins were not changed, whereas the phosphorylation of these proteins was significantly increased compared to the control group (see FIG. 20). In FIG. 20, control and MSS represent the control group and the MSM₁₈₀SS₂₃₀Sp₅₀ group, respectively. This suggests that, in the group administered with MSS for 3 weeks, a memory-related signal transduction process was significantly activated to enhance synaptic plasticity. Also, this shows that the change in the signal transduction-related molecules of the test animals was consistent with the improvement of memory and learning functions.

### INDUSTRIAL APPLICABILITY

As described above, the inventive composition, comprising 4-carbon dicarboxylic acid and an emulsifying agent (and an alcohol extract of maesil(plum, MS) or a maesil concentrate) is continuously administered to test animals for a given period of time, it will provide an anti-depressant effect for the test animals and also effectively improve the spatial recognition learning and working memory of the test animals. Thus, the inventive composition can be used as an antidepressant composition and a composition for the improvement of learning and memory functions.

1. A composition for the improvement of memory and learning functions, comprising: one or more 4-carbon dicarboxylic acids selected from the group consisting of succinic acid, succinate salt, fumaric acid and fumarate salt; and an emulsifying agent.

2. The composition of claim 1, wherein the emulsifying agent is one or a mixture of two or more selected from the group consisting of Tween80 (polyoxyethylene sorbitan monooleate), Span80 (sorbitan monooleate), bile acid, glycerin, glycerin fatty acid ester, propylene glycol, sucrose, sorbitol, sodium cholate, sorbitan monolaurate, sorbitan monopalmitate, sorbitan tristearate, sorbitan monostearate, polyoxyethylene sorbitan monolaurate, polyoxyethylene sorbitan monopalmitate, polyoxyethylene sorbitan monostearate, deoxycholate, sodium taurocholate, lecithins and lysolecithins.

3. The composition of claim 1, wherein the weight ratio of the 4-carbon dicarboxylic acid to the emulsifying agent is 1:0.05-5.

4. The composition of claims 1, which additionally contains a maesil(plum) concentrate or an alcohol extract of maesil.

5. The composition of claim 4, wherein the maesil concentrate or the alcohol extract of maesil has a malic acid content of 3-15% and a fumaric acid content of 0.3-10%.

6. The composition of claim 4, wherein the weight ratio between the maesil concentrate or the alcohol extract of maesil to the 4-carbon dicarboxylic acid is 1:0.1-5.

7. The composition of claim 6, wherein the weight ratio between the maesil(plum) concentrate, sodium succinate and Span80 (sorbitan monooleate) is 1:1-2:0.2-1.

8. The composition of claim 2, which additionally contains a maesil(plum) concentrate or an alcohol extract of maesil.

9. The composition of claim 3, which additionally contains a maesil(plum) concentrate or an alcohol extract of maesil.

10. The composition of claim 8, wherein the maesil concentrate or the alcohol extract of maesil has a malic acid content of 3-15% and a fumaric acid content of 0.3-10%.

11. The composition of claim 9, wherein the maesil concentrate or the alcohol extract of maesil has a malic acid content of 3-15% and a fumaric acid content of 0.3-10%.

12. The composition of claim 10, wherein the weight ratio between the maesil concentrate or the alcohol extract of maesil to the 4-carbon dicarboxylic acid is 1:0.1-5.

13. The composition of claim 11, wherein the weight ratio between the maesil concentrate or the alcohol extract of maesil to the 4-carbon dicarboxylic acid is 1:0.1-5.

14. The composition of claim 12, wherein the weight ratio between the maesil(plum) concentrate, sodium succinate and Span80 (sorbitan monooleate) is 1:1-2:0.2-1.

15. The composition of claim 13, wherein the weight ratio between the maesil(plum) concentrate, sodium succinate and Span80 (sorbitan monooleate) is 1:1-2:0.2-1.
